(19) United States
(12) Patent Application Publication
(10) Pub. No.: US 2025/0025409 A1
(43) Pub. Date: Jan. 23, 2025

(54) READY-TO-INFUSE PHENYLEPHRINE COMPOSITIONS

(71) Applicant: AMNEAL EU, LIMITED, Tipperary County (IE)

(72) Inventors: Piyush Amrutlal Patel, Ahmedabad (IN); Chiragkumar Girishbhai Siddhapara, Ahmedabad (IN); Aditya Maheshchandra Patel, Ahmedabad (IN); Amarender Reddy Donthidi, Hyderabad (IN); Srinivas Ramachandra Rao Kone, Monroe Township, NJ (US)

(73) Assignee: AMNEAL EU, LIMITED, Tipperary County (IE)

(21) Appl. No.: 18/243,271

(22) Filed: Sep. 7, 2023

(30) Foreign Application Priority Data
Jul. 14, 2023 (IN) IN202321047602

(51) Int. Cl. A61K 9/00; A61J 1/14; A61K 9/08; A61K 31/137; A61K 47/12

(52) U.S. Cl. CPC A61K 9/0019; A61J 1/1468; A61K 9/08; A61K 31/137; A61K 47/12

(57) ABSTRACT

The present invention relates to stable ready-to-infuse sulfite free compositions of phenylephrine or a pharmaceutically acceptable salt thereof and methods for preparing the same.

### RELATED APPLICATIONS

This application claims priority to Indian patent application No. 202321047602, filed on Jul. 14, 2023.

### FIELD OF INVENTION

The present invention relates to stable ready-to-infuse sulfite free compositions of phenylephrine or a pharmaceutically acceptable salt thereof and methods for preparing the same.

### BACKGROUND OF INVENTION

Phenylephrine is an alpha-1 adrenergic receptor agonist indicated for increasing blood pressure in adults with hypotension resulting primarily from vasodilation, in settings of septic shock and anesthesia. Phenylephrine is a strong vasoconstrictor used as a vasopressor to treat hypotension during anesthesia and septic shock; and frequently used in emergencies to treat hemodynamically unstable patients, who need the medication emergently.

Phenylephrine, chemically known as (R)-3-Hydroxy-α-(methylaminomethyl)benzyl alcohol, has the following structural formula.

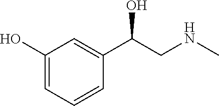

Phenylephrine is currently marketed as a concentrated intravenous solution, 10 mg/ml formulation (50 mg/5 mL and 100 mg/10 mL). However, the solution requires dilution in saline or other diluents prior to use as an intravenous bolus or for continuous intravenous infusion. The label for the currently marketed product indicates that the diluted solution should not be held for more than 4 hours at room temperature or for more than 24 hours under refrigerated conditions (2° C.-8° C.).

Depending upon the fluid restrictions to avoid volume overload, phenylephrine can be delivered to patients from a concentration of about 20 mcg/mL to about 400 mcg/mL (for central lines only). The necessity to dilute commercial formulations introduces a possibility of dosing errors, either from under dilution or over-dilution. Furthermore, transferring patients from one hospital setting to another presents the opportunity for treatment errors when to-be-delivered concentrations differ between the clinical settings. Finally, hospitals always struggle to compound this medication and get it to the bedside in a timely manner. Before outsourcing facilities existed, hospital clean rooms had the burden of compounding all phenylephrine drips in variable concentrations. Dilution steps for concentrated commercial drug products such as phenylephrine have been long recognized to contribute to the major problem of drug administration errors.

American Society of Health-System Pharmacists (ASHP) recommends a phenylephrine first line concentration of 80 mcg/mL and a second line concentration of 400 mcg/mL. The 80 mcg/ml concentration is particularly useful for managing vasodilatory shock, one of the clinical indications approved for marketed phenylephrine hydrochloride injection.

Further, the marketed phenylephrine hydrochloride injection 10 mg/mL contains antioxidant sodium metabisulfite, a sulfite that may cause allergic-type reactions, including anaphylactic symptoms and life-threatening or less severe asthmatic episodes in certain susceptible people.

Hence, there is need for sulfite free, ready-to-infuse composition of phenylephrine, e.g., phenylephrine hydrochloride, to relieve the burden of compounding at site and provide improved sterility and safety component to hospital pharmacies, with obvious benefit of reducing the risk of drug administration errors.

### SUMMARY OF THE INVENTION

The present invention relates to an aqueous ready-to-infuse phenylephrine injection solution comprising a dose amount of phenylephrine or a pharmaceutically acceptable salt thereof, and a citrate buffer in an amount of from about 0.0001 mg/mL to about 0.15 mg/mL. The solution upon storage for at least about 6 months at 25° C.±2° C./40%±5% relative humidity does not contain more than 0.2% of phenylephrine citrate adduct impurity, based on the dose amount of the phenylephrine or a pharmaceutically acceptable salt thereof.

In another embodiment, the present invention relates to an aqueous ready-to-infuse phenylephrine injection solution comprising a dose amount of phenylephrine or a pharmaceutically acceptable salt thereof, and a citrate buffer in an amount of from about 0.0001 mg/mL to about 0.15 mg/mL. The solution upon storage for at least about 6 months at 40° C.±2° C./not more than 25% relative humidity does not contain more than 0.2% of phenylephrine citrate adduct impurity, based on the dose amount of the phenylephrine or a pharmaceutically acceptable salt thereof.

In another embodiment, the present invention relates to an aqueous ready-to-infuse phenylephrine injection solution comprising a dose amount of phenylephrine or a pharmaceutically acceptable salt thereof and a citrate buffer in an amount of from about 0.0001 mg/ml to about 0.15 mg/mL. The solution, when subjected to at least one autoclave cycle for about 15 minutes, contains a phenyl citrate adduct impurity in an amount of not more than 0.2% of the dose amount of the phenylephrine or a pharmaceutically acceptable salt thereof.

In another embodiment, the present invention relates to an aqueous ready-to-infuse phenylephrine injection solution comprising a dose amount of phenylephrine or a pharmaceutically acceptable salt thereof, and a citrate buffer in an amount of from about 0.0001 mg/mL to about 0.15 mg/mL. The solution, upon storage for at least about 6 months at 25° C.±2° C./40%±5% relative humidity, exhibits an osmolality of from about 260 mOsmol/Kg to about 330 mOsmol/Kg.

In another embodiment, the present invention relates to an aqueous ready-to-infuse phenylephrine injection solution comprising a dose amount of phenylephrine or a pharmaceutically acceptable salt thereof, and a citrate buffer in an amount of from about 0.0001 mg/mL to about 0.15 mg/mL. The solution upon storage for at least about 6 months at 40° C.±2° C./not more than 25% relative humidity, exhibits an osmolality of from about 260 mOsmol/Kg to about 330 mOsmol/Kg.

In another embodiment, the present invention relates to an aqueous ready-to-infuse phenylephrine injection solution comprising a dose amount of phenylephrine or a pharmaceutically acceptable salt thereof, and a citrate buffer in an amount of from about 0.0001 mg/mL to about 0.15 mg/mL. The solution, upon storage for at least about 6 months at 25° C.±2° C./40%±5% relative humidity, contains a total impurity of not more than 0.8% of the dose amount of the phenylephrine or a pharmaceutically acceptable salt thereof.

In another embodiment, the present invention relates to an aqueous ready-to-infuse phenylephrine injection solution comprising a dose amount of phenylephrine or a pharmaceutically acceptable salt thereof and a citrate buffer in an amount of from about 0.0001 mg/mL to about 0.15 mg/mL. The solution, upon storage for at least about 6 months at 40° C.±2° C./not more than 25% relative humidity, contains a total impurity of not more than 0.8% of the dose amount of the phenylephrine or a pharmaceutically acceptable salt thereof.

In another embodiment, the present invention relates to an aqueous ready-to-infuse phenylephrine injection solution comprising a dose amount of phenylephrine or a pharmaceutically acceptable salt thereof and a citrate buffer in an amount of from about 0.0001 mg/ml to about 0.15 mg/mL. The solution, when subjected to at least one autoclave cycle for about 15 minutes, contains a total impurity of not more than 0.8% of the dose amount of the phenylephrine or a pharmaceutically acceptable salt thereof.

In certain embodiments, the injection solution comprises from about 0.01 mg/mL to about 1 mg/mL of phenylephrine hydrochloride.

In certain embodiments, the citrate buffer comprises sodium citrate and citric acid.

In certain embodiments, the pH adjusting agent is an acidic pH adjusting agent and a basic pH adjusting agent.

In certain embodiments, the acidic pH adjusting agent is hydrochloric acid.

In certain embodiments, the basic pH adjusting agent is sodium hydroxide.

In certain embodiments, the solution, upon storage for at least about 6 months at 25° C.±2° C./40%±5% relative humidity, maintains a pH of from about 3.0 to about 6.5.

In certain embodiments, the solution is free of sodium metabisulfite.

In certain embodiments, the solution is free of disodium edetate.

In certain embodiments, the solution is free of sodium metabisulfite and disodium edetate.

In another embodiment, the invention relates to an aqueous ready-to-infuse phenylephrine injection solution supplied in a flexible plastic container packed in aluminium over pouch, and an oxygen scavenger between the bag and the aluminum over pouch. The solution comprises phenylephrine or a pharmaceutically acceptable salt thereof, a pH adjusting agent, and a citrate buffer in an amount of from about 0.0001 mg/mL to about 0.15 mg/mL. The solution is free of antioxidant and disodium edetate.

In certain embodiments, the solution comprises from about 0.01 mg/mL to about 1 mg/mL of phenylephrine hydrochloride.

In certain embodiments, the citrate buffer comprises sodium citrate and citric acid.

In certain embodiments, the pH adjusting agent is an acidic pH adjusting agent and a basic pH adjusting agent.

In certain embodiments, the aqueous ready-to-infuse phenylephrine injection solutions of the disclosure are suitable for single use.

In certain embodiments, the phenylephrine salt is selected from the group consisting of phenylephrine hydrochloride, phenylephrine bitartrate, phenylephrine tartrate, phenylephrine diacetate hydrochloride, phenylephrine palmitate, and phenylephrine tetraphenyl borate.

In certain embodiments, the ready-to-infuse compositions comprise from about 0.01 mg/mL to about 1 mg/mL of phenylephrine hydrochloride. In certain embodiments, the ready-to-infuse compositions comprise from about 0.08 mg/mL to about 0.4 mg/mL of phenylephrine hydrochloride.

In certain embodiments, the pH adjusting agent is selected from the group consisting of acetic acid, citric acid, hydrochloric acid, phosphoric acid, sodium hydroxide, and mixtures thereof. In certain embodiments, the pH adjusting agent is a mixture of hydrochloric acid and sodium hydroxide. In certain embodiments, the composition maintains a pH of from about 3.0 to about 6.5. In certain embodiments, the composition maintains a pH of from about 4.0 to about 5.0.

In certain embodiments, the aqueous ready-to-infuse solution further comprises a tonicity agent selected from the group consisting of sodium chloride, dextrose solution, ringer's lactate solution, and mixtures thereof. In certain embodiments, the tonicity agent is 0.9% sodium chloride. In certain embodiments, the tonicity agent is 5% dextrose.

In certain embodiments, the aqueous ready-to-infuse phenylephrine solution comprises a fill volume of from about 100 mL to about 500 mL. In certain embodiments, the solution comprises a fill volume of about 250 mL.

In certain embodiments, the flexible plastic container is a bag.

In certain embodiments, the solution is terminally sterilized.

Further, the present invention relates to a method for making a stable ready-to-infuse pharmaceutical composition of phenylephrine or a pharmaceutically acceptable salt thereof. The method comprises: 1) adding a tonicity agent to about 95% of the total volume of water for injection (WFI) and mixing to obtain a clear solution: 2) adding buffer to the solution from step #1 and mixing to obtain a clear solution: 3) adding phenylephrine hydrochloride to the solution from step #2 and mixing to obtain a clear solution: 4) adding a pH adjusting agent to the solution from step #3 to obtain a pH of about 4.5 and mixing to obtain a pH adjusted solution: 5) adding remaining amount of WFI to the pH adjusted solution from step #4 to make up the volume to 100% and mixing to obtain a bulk solution: 6) filtering the bulk solution from step #5 to obtain a filtered solution: 7) filing the filtered solution from step #6 into a bag and stoppered with a twist off port: 8) terminally sterilizing the bag from step #7 to obtain a terminally sterilized product: 9) placing an oxygen scavenger on the bag from step #8 and overwrapping with an aluminum over pouch.

### DETAILED DESCRIPTION OF INVENTION

The present disclosure provides stable ready-to-infuse sulfite free phenylephrine compositions that are free of any chelating agent and comprise buffer in an amount of less than about 0.15 mg/ml. The compositions maintain their stability under long-term, intermediate, and accelerated storage conditions for at least about 6 months.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the word “a” or “an” when used in conjunction with the term “comprising” in the claims and/or the specification can mean “one,” but it is also consistent with the meaning of “one or more,” “at least one,” and “one or more than one.” Still further, the terms “having,” ‘including,” “containing,” or “comprising” are interchangeable, and one of skill in the art is cognizant that these terms are open-ended terms.

As used herein, the terms “comprise,” “comprises”, and “comprising” are to be interpreted inclusively rather than exclusively. The words “consist,” “consisting,” and its variants, are to be interpreted exclusively, rather than inclusively.

As used herein, the term “and/or” refers to and encompasses any and all possible combinations of one or more of associated listed items.

As used herein, and unless otherwise specified, the terms “about” or “approximately” mean an acceptable error for a particular value as determined by one of ordinary skill in the art, which depends in part on how the value is measured or determined. In certain embodiments, the term “about” or “approximately” means within 5%, 4%, 3%, 2%, 1%, 0.5%, 0.1%, or 0.05% of a given value or range.

As used herein, and unless otherwise specified, the term “range” refers to values provided, and is intended to include each intervening value between the upper and lower limit of that range and any other stated or intervening values in that stated range encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

As used herein, unless otherwise specified, the term “stable” refers to stable injectable compositions containing from about 80% to about 120% of the labelled amount of phenylephrine or a pharmaceutically acceptable salt thereof.

As used herein, unless otherwise specified, the term “phenylephrine citrate adduct,” refers to N-citryl (R)-phenylephrine (also known as 2-Hydroxy-2-(2-{[(R)-2-hydroxy-2-(3-hydroxyphenyl)ethyl](methyl)amino}-2-oxoethyl)succinic acid).

The terms “dose amount,” “labelled amount,” and “initial assay,” as used interchangeably herein, refer to the amount of drug, as mentioned on drug prescribing information, that is administered during a single use of the ready-to-infuse injection composition.

The terms “room temperature” and “controlled room temperature,” as used interchangeably herein, refer to 20° C. to about 25° C.

As used herein, the term “long-term storage conditions” refers to storage at 25° C.±2° C./40%±5% relative humidity.

As used herein the term “accelerated storage conditions” refers to storage at 40° C.±2° C./not more than 25% relative humidity.

As used herein the term “intermediate storage conditions” refers to storage at 30° C.±2° C./65%±5% relative humidity.

As used herein the term “refrigerated storage conditions” refers to storage at 2-8° C.

As used herein the term “freezing storage conditions” refers to storage at −20° C.±5° C.

As used herein the term “aqueous” should be accorded its normal meaning which may mean the composition, formulation or material that contains water as a solvent or medium.

The terms “ready-to-use,” and ready-to-infuse,” as used interchangeably herein, refer to injectable compositions that do not require any further dilution or reconstitution prior to infusion in a patient in need thereof.

The terms “flexible plastic container “refers to a flexible bag or a flexible bottle.

As used herein, and unless otherwise specified, the term “terminal sterilization” is defined as the process whereby product is sterilized within its sterile barrier system. The terminal sterilization process is considered a manufacturing process step itself and usually takes place at, or near, the end of the manufacturing process. An autoclave is commonly used to accomplish terminal heat sterilization of drug products in their final packaging. Typical autoclave cycles in the pharmaceutical industry to achieve terminal sterilization of the final product involve heating to about 121° C. for a predefined time and cycles (e.g., 30-90 minutes and 4 cycles or less).

As used herein, and unless otherwise specified, the term “pH adjusting agent” refers to acids and bases that are added to control acidity or alkalinity of the ready-to-infuse compositions of the disclosure.

As used herein, and unless otherwise specified, the term “buffering agent” refers to a weak acid together with one of its salts or alkali together with one of its salts, which resists changes in pH (e.g., maintains pH) of the ready-to-infuse compositions of the disclosure.

As used herein, and unless otherwise specified, the term “shelf-life” refers to a period during which a pharmaceutical composition may be stored and remains suitable for use. The shelf-life of the ready-to-infuse injectable compositions of the disclosure is about 24 months.

The terms “chelating agents” and “chelators,” as used interchangeably herein, refer to multi-dentate compounds that complex metal cations and they have no other significant physiological role in a mammal. Therefore, EDTA is deemed a metal ion chelator whereas citrate or tartrate are not deemed a metal chelator.

The term “antioxidant” refers to chemical compounds that reduce the oxidation of active substances and excipients in the finished product. It inhibits the oxidation by scavenging reactive oxygen species (ROS) that are produced as by-products.

### Phenylephrine Hydrochloride Injection Compositions

The present invention relates to stable ready-to-infuse injection compositions of phenylephrine or a pharmaceutically acceptable salt thereof and methods for preparing the same. In certain embodiments, the ready-to-infuse phenylephrine compositions of the disclosure are suitable for single use only. In particular, the invention relates to stable ready-to-infuse injection compositions comprising phenylephrine or a pharmaceutically acceptable salt thereof, a buffer, a pH adjusting agent, and a tonicity agent. In certain embodiments, the ready-to-infuse phenylephrine compositions of the disclosure comprise intravenous injections suitable for intravenous administration.

In certain embodiments, the ready-to-infuse injection compositions of the disclosure comprise a phenylephrine salt selected from the group consisting of phenylephrine hydrochloride, phenylephrine bitartrate, phenylephrine tartrate, phenylephrine diacetate hydrochloride, phenylephrine palmitate, and phenylephrine tetraphenyl borate. In certain embodiments, the compositions comprise phenylephrine hydrochloride.

In certain embodiments, the ready-to-infuse injection compositions of the disclosure comprise from about 0.01 mg/mL to about 1 mg/mL of phenylephrine hydrochloride. In certain embodiments, the compositions comprise about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/ml, about 0.9 mg/ml, about 1 mg/ml, or any intermediate values therein. In certain embodiments, the compositions comprise about 0.08 mg/mL (20 mg/250 mL), about 0.1 mg/mL (25 mg/250 mL), about 0.16 mg/mL (40 mg/250 mL) or about 0.4 mg/mL (100 mg/250 mL) of phenylephrine hydrochloride.

In certain embodiments, the ready-to-infuse phenylephrine compositions of the disclosure comprise at least one buffer comprising a weak acid with one of its salts or an alkali together with one of its salts, which resists changes in pH (e.g., maintains pH) of composition.

In certain embodiments, the buffer is selected from the group consisting of citrate buffer, phosphate buffer, acetate buffer, tartrate buffer, glutamate buffer, triethanolamine (TRIS) buffer, and mixtures thereof. In certain embodiments, the compositions comprise citrate buffer comprising citric acid and its salt. In certain embodiments, the citrate buffer further comprises citric acid and sodium citrate.

In certain embodiments, the ready-to-infuse phenylephrine compositions of the disclosure comprise buffer from about 0.0001 mg/mL to about 0.2 mg/mL, from about 0.0001 mg/ml to about 0.19 mg/ml, from about 0.0001 mg/ml to about 0.18 mg/ml, from about 0.0001 mg/ml to about 0.17 mg/ml, from about 0.0001 mg/ml to about 0.16 mg/ml, from about 0.0001 mg/ml to about 0.15 mg/ml, from about 0.0001 mg/ml to about 0.14 mg/ml, from about 0.0001 mg/ml to about 0.13 mg/ml, from about 0.0001 mg/ml to about 0.12 mg/ml, from about 0.0001 mg/ml to about 0.11 mg/ml, or from about 0.0001 mg/ml to about 0.1 mg/ml, or any intermediate values therein. In certain embodiments, the ready-to-infuse phenylephrine compositions of the disclosure comprise buffer in an amount of about 0.0001 mg/mL, about 0.0005 mg/mL, about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/ml, about 0.04 mg/mL, about 0.05 mg/mL, about 0.06 mg/mL, about 0.07 mg/mL, about 0.08 mg/mL, about 0.09 mg/mL, about 0.1 mg/mL, about 0.15 mg/mL, about 0.2 mg/mL, or any intermediate values therein. In certain embodiments, the total amount of buffer includes the amount of weak acid or alkali together with one of its salts. In certain embodiments, the ready-to-infuse phenylephrine compositions of the disclosure comprise from about 0.0001 mg/mL to about 0.15 mg/mL of citrate buffer. In certain embodiments, the ready-to-infuse phenylephrine compositions of the disclosure comprise citrate buffer in an amount of from about 0.0001 mg/mL to about 0.2 mg/mL, from about 0.0001 mg/ml to about 0.19 mg/ml, from about 0.0001 mg/ml to about 0.18 mg/ml, from about 0.0001 mg/ml to about 0.17 mg/ml, from about 0.0001 mg/ml to about 0.16 mg/ml, from about 0.0001 mg/ml to about 0.15 mg/ml, from about 0.0001 mg/ml to about 0.14 mg/ml, from about 0.0001 mg/ml to about 0.13 mg/ml, from about 0.0001 mg/ml to about 0.12 mg/ml, from about 0.0001 mg/ml to about 0.11 mg/ml, or from about 0.0001 mg/ml to about 0.1 mg/ml, or any intermediate values therein. In certain embodiments, the ready-to-infuse phenylephrine compositions of the disclosure comprise citrate buffer in an amount of about 0.0001 mg/mL, about 0.0005 mg/mL, about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/ml, about 0.04 mg/mL, about 0.05 mg/mL, about 0.06 mg/mL, about 0.07 mg/mL, about 0.08 mg/mL, about 0.09 mg/mL, about 0.1 mg/mL, about 0.15 mg/mL, or any intermediate values therein. In certain embodiments, the citrate buffer comprises citric acid and sodium citrate. In certain embodiments, the citrate buffer comprises 0.016 mg/ml of citric acid and 0.064 mg/ml of sodium citrate.

In certain embodiments, the ready-to-infuse phenylephrine compositions of the disclosure comprise at least one pH adjusting agent comprising at least one acid and/or at least one base to control acidity or alkalinity of the compositions. In certain embodiments, the ready-to-infuse phenylephrine compositions of the disclosure comprise at least one pH adjusting agent to provide a pH of from about 3.0 to about 6.5. In certain embodiments, the pH adjusting agent is selected from the group consisting of acetic acid, citric acid, hydrochloric acid, phosphoric acid, sodium hydroxide, and mixtures thereof. In certain embodiments, the pH adjusting agent is a mixture of hydrochloric acid and sodium hydroxide.

In certain embodiments, the pharmaceutical composition of phenylephrine or a pharmaceutically acceptable salt thereof exhibits a pH range of from about 3.0 to about 6.5. In certain embodiments, the composition exhibits a pH of from about 3.1 to about 6.4, about 3.2 to about 6.3, about 3.3 to about 6.2, about 3.4 to about 6.1, about 3.5 to about 6.0, or any intermediate values therein. In certain embodiments, the composition exhibits a pH of from about 4.0 to about 5.0. In certain embodiments, the compositions exhibit a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5 or any intermediate values therein.

In certain embodiments, the ready-to-infuse phenylephrine compositions of the disclosure comprise at least one tonicity agent selected from the group consisting of sodium chloride, potassium chloride, dextrose solution, sugars, sugar alcohols, glucose, xylitol, fructose, glycerol, sorbitol, mannitol, mannose, calcium chloride, magnesium chloride, other inorganic salts, ringer's lactate solution and mixtures thereof. In certain embodiments, the tonicity agent is sodium chloride. In certain embodiments, the tonicity agent is present in an amount of from about 5 mg/mL to about 20 mg/mL, from about 5 mg/mL to about 10 mg/mL, about 6 mg/mL, about 7 mg/mL, about 8 mg/mL, about 9 mg/mL, or any intermediate values therein. In certain embodiments, the tonicity agent is 0.9% sodium chloride (9 mg/mL). In certain embodiments, the tonicity agent is 5% dextrose (50 mg/mL).

In certain embodiments, the ready-to-infuse phenylephrine compositions of disclosure are supplied in a fill volume of from about 100 mL to about 500 mL. In certain embodiments, the ready-to-infuse phenylephrine compositions are supplied in a fill volume of about 100 ml, about 150 ml, about 200 mL, about 250 mL, about 300 mL, about 350 mL, about 400 mL, about 450 mL, about 500 mL, or any intermediate volumes therein. In certain embodiments, the ready-to-infuse phenylephrine compositions are supplied in a fill volume of about 100 mL.

In certain embodiments, the composition is supplied in the fill volume of about 250 mL. In certain embodiments, the ready-to-infuse phenylephrine compositions of the disclosure are supplied in flexible plastic containers as opposed to rigid or semi-rigid containers. In certain embodiments, the flexible plastic container is a flexible bag or a flexible bottle.

In certain embodiments, the flexible plastic container is a closed system fully collapsible intravenous bag. The bag provides maximum drainage and minimum residual volume of the infusion liquid to minimize dosing error. Additionally, the bag does not require external venting to empty the bag.

In certain embodiments, the flexible plastic container is further packed in a secondary packaging material. In certain embodiments, secondary packaging material is an aluminum overwrap, aluminum over pouch or equivalent thereof. In certain embodiments, secondary packaging material is an aluminum over pouch.

In certain embodiments, the ready-to-infuse phenylephrine compositions of the disclosure are stabilized by minimizing the oxygen level in the composition. In certain embodiments, the oxygen level in the composition is minimized by manufacturing the injection composition in an inert atmosphere comprising an inert gas, e.g., nitrogen gas, to flush out any oxygen. In certain embodiments, the oxygen level in the composition is minimized by including at least one oxygen scavenger/oxygen absorber or by flushing an inert gas between the flexible plastic container and the aluminum over pouch. In certain embodiments, the oxygen scavenger comprises an oxygen absorbing material in a packet or a sachet. In certain embodiments, an oxygen scavenger/oxygen absorber packet/sachet is placed between the flexible bag and the aluminum over pouch to remove or minimize the oxygen level in the packaging and control and minimize drug degradation. In certain embodiments, the oxygen scavenger is packed in a porous sachet or packet. Commercially available oxygen scavengers used in the ready-to-infuse phenylephrine compositions of the disclosure include, but are not limited to, Standa ATCO®, ATCO® HV 210/100, GLS100, PHARMAKEEP®: Mitsubishi AGELESS®. AGELESS® SS-MBC, AGELESS® ZPT-100MBC, AGELSEE® ZH-100, D Series FRESHPAX™, FRESHMAX®, STABILOX® D-200-H60 and STABILOX® D-100-H75.

In certain embodiments, the space between the flexible plastic container and the secondary packaging material is filled with inert gas. An inert gas may be used to flush or replace the air from the space between the flexible plastic container and the secondary packaging material. The inert gases that may be used include nitrogen, argon, and helium. In certain embodiments, the inert gases that may be nitrogen.

In certain embodiments, the dissolved oxygen content of pharmaceutical composition of present invention is maintained at not more than (NMT) 2 ppm. In certain embodiments, the dissolved oxygen content of pharmaceutical composition of present invention is maintained at less than 2 ppm, less than 1.5 ppm, less than 1 ppm, less the 0.5 ppm, or any intermediate values therein.

Commercially available intravenous bags used in the ready-to-infuse phenylephrine compositions of the disclosure include, but are not limited to EXCEL®, VISIV®, NEXCEL®, INTERVIA®, TECHNOFLEX®, SOLOMIX®, STEDIM71®, STEDIM®100, VIAFLEX®, VIAFLO®, ADDEASE®, ADD-VANTAGE®, DUPLEX®, FIRST CHOICE®, PROPYFLEX®, FREEFLEX®, POLYCINE®, MAGIFLEX® and BFS®.

In certain embodiments, the flexible plastic container is flexible bag comprising a non-plasticized film containing polypropylene and thermoplastic elastomers (FREEFLEX®).

In certain embodiments, the flexible plastic container is a flexible bag comprising a multilayer polyolefin/styrene block copolymer-based film (POLYCINE®).

In certain embodiments, the flexible plastic container is a flexible bag comprising a multilayer sheeting composed of Polypropylene, Polyamide and Polyethylene (VIAFLO®).

In certain embodiments, the flexible plastic container is a flexible bag comprising a triple-layer double-wound PVC-free (Polypropylene based) film (MAGIFLEX®).

In certain embodiments, the flexible plastic container is a flexible bag comprising a multilayer film comprising polypropylene polymer or polypropylene based polyolefin polymer and styrene-ethylene-butylene block copolymer (TECHNOFLEX®).

In certain embodiments, the flexible plastic container is a flexible bag which is a non-DEHP (Di-ethylhexyl-phthalate), non-PVC (polyvinyl chloride) and latex-free.

In certain embodiments, the ready-to-infuse phenylephrine compositions of the disclosure are free of a commonly used chelating agent disodium edetate.

In certain embodiments, the ready-to-infuse phenylephrine compositions of the disclosure are free of antioxidant selected from the group consisting of alpha tocopherol, ascorbic acid, butylated hydroxy anisole, butylated hydroxytoluene, methionine, monothioglycerol, potassium metabisulfite, propyl gallate, sodium ascorbate, sodium bisulfite, sodium metabisulfite, sodium sulfite, sodium thiosulfate, salts and hydrates thereof, and any mixtures thereof. In certain embodiments, the composition is free of sodium metabisulfite.

In certain embodiments, the ready-to-infuse phenylephrine compositions of the disclosure are free of preservatives, e.g., benzyl alcohol, phenol, parabens, etc.

In certain embodiments, the ready-to-infuse phenylephrine compositions of the disclosure are terminally sterilized using typical autoclave cycles used in the pharmaceutical industry for terminal sterilization of the final product. In certain embodiments, the ready-to-infuse phenylephrine compositions of the disclosure are autoclaved at about 121° C. for about 15 minutes, 121° C. for about 30 minutes, 121° C. for about 60 minutes or more, based on F₀ based cycle, to assure sterility assurance of the finished product. In certain embodiments, the ready-to-infuse phenylephrine compositions of the disclosure are autoclaved at about 121° C. using steam-air mixture for at least about 15 minutes (e.g., about 20 minutes).

In certain embodiments, the ready-to-infuse phenylephrine compositions of the disclosure exhibit osmolality of from about 250 mOsmol/kg to about 450 mOsmol/kg. In certain embodiments, the compositions exhibit osmolality of from about 260 mOsmol/kg to about 330 mOsmol/kg, from about 270 mOsmol/kg to about 320 mOsmol/kg, from about 280 mOsmol/kg to about 310 mOsmol/kg. In certain embodiments, the composition exhibit osmolality of about 260, about 265, about 270, about 275, about 280, about 281, about 282, about 283, about 284, about 285, about 286, about 287, about 288, about 289, about 290, about 291, about 292, about 293, about 294, about 295, about 296, about 297, about 298, about 299, about 300, about 305, about 310, about 315, about 320, about 325, about 330 mOsmol/kg, or any intermediate values therein.

In certain embodiments, osmolality of ready-to-infuse phenylephrine compositions is determined by measuring the depression in freezing point of the injection solution using an osmometer. The osmometer comprises a means of cooling the container used for the measurement: a resistor sensitive to temperature (thermistor), with an appropriate current or potential difference measurement device that may be graduated in temperature change or in osmolality and a means of mixing the sample. The sample solution is prepared by taking a sample bag, shaking the bag to shake the contents well and withdrawing about 5 mL of sample into suitable clean and dried glass container as per sample withdrawal procedure. About 0.25 mL of sample solution is transferred into osmometer cuvette and osmolality is measured and recorded in mOsmol/kg.

In certain embodiments, the ready-to-infuse phenylephrine compositions of the disclosure are stable under long-term storage conditions for at least about 6 months. In certain embodiments the long-term storage conditions comprise storage of the ready-to-infuse phenylephrine composition in a bag with aluminum over pouch and oxygen scavenger between the bag and the aluminum over pouch at 25° C.±2° C./40% RH±5% RH. In certain embodiments, the ready-to-infuse phenylephrine compositions of the disclosure are stable under long-term storage conditions for at least about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months, about 23 months, about 2 years, about 30 months, about 3 years, about 4 years, about 5 years, or any intermediate periods therein. In certain embodiments, the ready-to-infuse phenylephrine compositions of the disclosure are stable under long-term storage conditions for at least about 12 months. In certain embodiments, the ready-to-infuse phenylephrine compositions of the disclosure are stable under long-term storage conditions for up to 36 months (3 years).

In certain embodiments, the ready-to-infuse phenylephrine compositions comprising a dose amount of phenylephrine or a pharmaceutically acceptable salt thereof, on storage under long-term storage conditions, comprise phenylephrine assay of from about 80% to about 120% of the dose amount. In certain embodiments, the phenylephrine compositions of the disclosure, on storage under long-term storage conditions, comprise phenylephrine assay of from about 80-120%, from about 85-115%, from about 90 to 110%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, or any intermediate values therein of the dose amount.

In certain embodiments, the ready-to-infuse phenylephrine compositions, on storage under long-term storage conditions, exhibit osmolality of from about 250 mOsmol/Kg to about 450 mOsmol/Kg. In certain embodiments, the composition exhibit osmolality of from about 260 mOsmol/kg to about 330 mOsmol/kg, from about 270 mOsmol/kg to about 320 mOsmol/kg, from about 280 mOsmol/kg to about 310 mOsmol/kg. In certain embodiments, the composition exhibit osmolality of about 280, about 281, about 282, about 283, about 284, about 285, about 286, about 287, about 288, about 289, about 290, about 291, about 292, about 293, about 294, about 295, about 296, about 297, about 298, about 299, about 300, or any intermediate values therein.

In certain embodiments, the ready-to-infuse phenylephrine compositions comprising a dose amount of phenylephrine or a pharmaceutically acceptable salt thereof, on storage under long-term storage conditions, comprise a total impurity of not more than (NMT) about 0.8% of the dose amount of the phenylephrine or a pharmaceutically acceptable salt thereof. In certain embodiments, the compositions comprise a total impurity of NMT 0.8%, 0.7%, 0.6%, 0.5%, 0.4%, 0.3%, 0.2%, 0.1%, 0.05%, or any intermediate values therein, of the dose amount.

In certain embodiments, the compositions comprise a total impurity of NMT about 0.2%. In certain embodiments, the compositions comprise a total impurity of NMT 0.1% of the dose amount.

In certain embodiments, the ready-to-infuse phenylephrine compositions comprising a dose amount of phenylephrine or a pharmaceutically acceptable salt thereof, on storage under long-term storage conditions, comprise phenylephrine citrate adduct impurity of not more than (NMT) about 0.2% of the dose amount of the phenylephrine or a pharmaceutically acceptable salt thereof. In certain embodiments, the compositions comprise phenylephrine citrate adduct impurity of NMT 0.1%, 0.05%, 0.01%, 0.001%, or any intermediate values therein. In certain embodiments, no phenylephrine citrate adduct impurity is determined after storage of the product under long-term storage conditions.

In certain embodiments, the ready-to-infuse phenylephrine compositions of the disclosure are stable under accelerated storage conditions for at least about 6 months. In certain embodiments the accelerated storage conditions comprise storage of the ready-to-infuse phenylephrine composition in a bag with aluminum over pouch and oxygen scavenger between the bag and the aluminum over pouch at 40° C.±2° C./NMT25% RH. In certain embodiments, the ready-to-infuse phenylephrine compositions of the disclosure are stable under long-term storage conditions for at least about 6 months, about 7 months, about 8 months, about 9 months, about 10 months, about 11 months, about 1 year, or any intermediate periods therein. In certain embodiments, the ready-to-infuse phenylephrine compositions of the disclosure are stable under accelerated storage conditions for at least about 6 months or about 12 months.

In certain embodiments, the ready-to-infuse phenylephrine compositions comprising a dose amount of phenylephrine or a pharmaceutically acceptable salt thereof, on storage under accelerated storage conditions, comprise phenylephrine assay of from about 80% to about 120% of the dose amount. In certain embodiments, the phenylephrine compositions of the disclosure, on storage under accelerated storage conditions, comprise phenylephrine assay of from about 80-120%, from about 85-115%, from about 90 to 110%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, or any intermediate values therein of the dose amount.

In certain embodiments, the ready-to-infuse phenylephrine compositions, on storage under accelerated storage conditions, exhibit osmolality of from about 250 mOsmol/Kg to about 450 mOsmol/Kg. In certain embodiments, the composition exhibit osmolality of from about 260 mOsmol/kg to about 330 mOsmol/kg, from about 270 mOsmol/kg to about 320 mOsmol/kg, from about 280 mOsmol/kg to about 310 mOsmol/kg. In certain embodiments, the composition exhibit osmolality of about 280, about 281, about 282, about 283, about 284, about 285, about 286, about 287, about 288, about 289, about 290, about 291, about 292, about 293, about 294, about 295, about 296, about 297, about 298, about 299, about 300, or any intermediate values therein.

In certain embodiments, the ready-to-infuse phenylephrine compositions comprising a dose amount of phenylephrine or a pharmaceutically acceptable salt thereof, on storage under accelerated storage conditions, comprise a total impurity of not more than (NMT) about 0.8% of the dose amount of the phenylephrine or a pharmaceutically acceptable salt thereof. In certain embodiments, the compositions comprise a total impurity of NMT 0.8%, 0.7%, 0.6%, 0.5%, 0.4%, 0.3%, 0.2%, 0.1%, 0.05%, or any intermediate values therein, of the dose amount. In certain embodiments, the compositions comprise a total impurity of NMT about 0.2%. In certain embodiments, the compositions comprise a total impurity of NMT 0.1% of the dose amount.

In certain embodiments, the ready-to-infuse phenylephrine compositions comprising a dose amount of phenylephrine or a pharmaceutically acceptable salt thereof, on storage under accelerated storage conditions, comprise phenylephrine citrate adduct impurity of not more than (NMT) about 0.2% of the dose amount of the phenylephrine or a pharmaceutically acceptable salt thereof. In certain embodiments, the compositions comprise phenylephrine citrate adduct impurity of NMT 0.1%, 0.05%, 0.01%, 0.001%, or any intermediate values therein. In certain embodiments, no phenylephrine citrate adduct impurity is determined after storage of the product under accelerated storage conditions.

In certain embodiments, the ready-to-infuse phenylephrine compositions of the disclosure are stable under refrigerated storage conditions for at least about 6 months. In certain embodiments the refrigerated storage conditions comprise storage of the ready-to-infuse phenylephrine composition in a bag with aluminum over pouch and oxygen scavenger between the bag and the aluminum over pouch storage at about 2-8° C.

In certain embodiments, the ready-to-infuse phenylephrine compositions of the disclosure are stable under refrigerated storage conditions for at least about 6 months, about 7 months, about 8 months, about 9 months, about 10 months, about 11 months, about 1 year, or any intermediate periods therein. In certain embodiments, the ready-to-infuse phenylephrine compositions of the disclosure are stable under refrigerated storage conditions for at least about 6 months or about 12 months.

In certain embodiments, the ready-to-infuse phenylephrine compositions comprising a dose amount of phenylephrine or a pharmaceutically acceptable salt thereof, on storage under refrigerated storage conditions, comprise phenylephrine assay of from about 80% to about 120% of the dose amount. In certain embodiments, the phenylephrine compositions of the disclosure, on storage under refrigerated storage conditions, comprise phenylephrine assay of from about 80-120%, from about 85-115%, from about 90 to 110%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, or any intermediate values therein of the dose amount.

In certain embodiments, the ready-to-infuse phenylephrine compositions, on storage under refrigerated storage conditions, exhibit osmolality of from about 250 mOsmol/Kg to about 450 mOsmol/Kg. In certain embodiments, the compositions exhibit osmolality of from about 260 mOsmol/kg to about 330 mOsmol/kg, from about 270 mOsmol/kg to about 320 mOsmol/kg, from about 280 mOsmol/kg to about 310 mOsmol/kg. In certain embodiments, the composition exhibit osmolality of about 280, about 281, about 282, about 283, about 284, about 285, about 286, about 287, about 288, about 289, about 290, about 291, about 292, about 293, about 294, about 295, about 296, about 297, about 298, about 299, about 300, or any intermediate values therein.

In certain embodiments, the ready-to-infuse phenylephrine compositions comprising a dose amount of phenylephrine or a pharmaceutically acceptable salt thereof, on storage under refrigerated storage conditions, comprise a total impurity of not more than (NMT) about 0.8% of the dose amount of the phenylephrine or a pharmaceutically acceptable salt thereof. In certain embodiments, the compositions comprise a total impurity of NMT 0.8%, 0.7%, 0.6%, 0.5%, 0.4%, 0.3%, 0.2%, 0.1%, 0.05%, or any intermediate values therein, of the dose amount. In certain embodiments, the compositions comprise a total impurity of NMT about 0.2%. In certain embodiments, the compositions comprise a total impurity of NMT 0.1% of the dose amount.

In certain embodiments, the ready-to-infuse phenylephrine compositions comprising a dose amount of phenylephrine or a pharmaceutically acceptable salt thereof, on storage under refrigerated storage conditions, comprise phenylephrine citrate adduct impurity of not more than (NMT) about 0.2% of the dose amount of the phenylephrine or a pharmaceutically acceptable salt thereof. In certain embodiments, the compositions comprise phenylephrine citrate adduct impurity of NMT 0.1%, 0.05%, 0.01%, 0.001%, or any intermediate values therein. In certain embodiments, no phenylephrine citrate adduct impurity is determined after storage of the product under refrigerated storage conditions.

In certain embodiments, the ready-to-infuse phenylephrine compositions of the disclosure are stable under freezing storage conditions for at least about 6 months. In certain embodiments the freezing storage conditions comprise storage of the ready-to-infuse phenylephrine composition in a bag with aluminum over pouch and oxygen scavenger between the bag and the aluminum over pouch, at −20° C.±5° C.

In certain embodiments, the ready-to-infuse phenylephrine compositions of the disclosure are stable under freezing storage conditions for at least about 6 months, about 7 months, about 8 months, about 9 months, about 10 months, about 11 months, about 1 year, or any intermediate periods therein. In certain embodiments, the ready-to-infuse phenylephrine compositions of the disclosure are stable under freezing storage conditions for at least about 6 months or about 12 months.

In certain embodiments, the ready-to-infuse phenylephrine compositions comprising a dose amount of phenylephrine or a pharmaceutically acceptable salt thereof, on storage under freezing storage conditions, comprise phenylephrine assay of from about 80% to about 120% of the dose amount. In certain embodiments, the phenylephrine compositions of the disclosure, on storage under freezing storage conditions, comprise phenylephrine assay of from about 80-120%, from about 85-115%, from about 90 to 110%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, or any intermediate values therein of the dose amount.

In certain embodiments, the ready-to-infuse phenylephrine compositions, on storage under freezing storage conditions, exhibit osmolality of from about 250 mOsmol/Kg to about 450 mOsmol/Kg. In certain embodiments, the compositions exhibit an osmolality of from about 260 mOsmol/kg to about 330 mOsmol/kg, from about 270 mOsmol/kg to about 320 mOsmol/kg, from about 280 mOsmol/kg to about 310 mOsmol/kg. In certain embodiments, the composition exhibit osmolality of about 280, about 281, about 282, about 283, about 284, about 285, about 286, about 287, about 288, about 289, about 290, about 291, about 292, about 293, about 294, about 295, about 296, about 297, about 298, about 299, about 300, or any intermediate values therein.

In certain embodiments, the ready-to-infuse phenylephrine compositions comprising a dose amount of phenylephrine or a pharmaceutically acceptable salt thereof, on storage under freezing storage conditions, comprise a total impurity of not more than (NMT) about 0.8% of the dose amount of the phenylephrine or a pharmaceutically acceptable salt thereof. In certain embodiments, the compositions comprise a total impurity of NMT 0.8%, 0.7%, 0.6%, 0.5%. 0.4%, 0.3%, 0.2%, 0.1%, 0.05%, or any intermediate values therein, of the dose amount. In certain embodiments, the compositions comprise a total impurity of not more than about 0.2%. In certain embodiments, the compositions comprise a total impurity of not more than 0.1% of the dose amount.

In certain embodiments, the ready-to-infuse phenylephrine compositions comprising a dose amount of phenylephrine or a pharmaceutically acceptable salt thereof, on storage under freezing storage conditions, comprise phenylephrine citrate adduct impurity of not more than about 0.2% of the dose amount of the phenylephrine or a pharmaceutically acceptable salt thereof. In certain embodiments, the compositions comprise phenylephrine citrate adduct impurity of not more than 0.1%, 0.05%, 0.01%, 0.001%, or any intermediate values therein. In certain embodiments, no phenylephrine citrate adduct impurity is determined after storage of the product under refrigerated storage conditions.

In certain embodiments, the disclosure provides stable ready-to-infuse phenylephrine compositions comprising less than 0.2 mg/mL buffer, e.g., 0.08 mg/mL. In certain embodiments, the ready-to-infuse phenylephrine compositions of the disclosure, comprising less than 0.2 mg/mL buffer/buffering agent, upon storage for at least about 6 months under long-term storage conditions at 25° C.±2° C./40±5% RH, exhibit an assay of phenylephrine or pharmaceutically acceptable salt thereof from about 90% to about 110% of the dose amount. In certain embodiments, such compositions, upon storage under long-term storage conditions comprise phenylephrine citrate product impurity of less than 0.2% of the dose amount and total impurity of less than 0.8% of the dose amount.

In certain embodiments, the disclosure provides stable ready-to-infuse phenylephrine compositions comprising less than 0.2 mg/mL buffer, e.g., 0.08 mg/mL. In certain embodiments, the ready-to-infuse phenylephrine compositions of the disclosure, comprising less than 0.2 mg/mL buffer/buffering agent, upon storage for at least about 6 months under accelerated storage conditions at 40° C.±2° C./NMT25% RH, exhibit an assay of phenylephrine or pharmaceutically acceptable salt thereof from about 90% to about 110% of the dose amount. In certain embodiments, such compositions, upon storage under accelerated storage conditions comprise phenylephrine citrate product impurity of less than 0.2% of the dose amount and total impurity of less than 0.8% of the dose amount.

In certain embodiments, the disclosure provides stable ready-to-infuse phenylephrine compositions comprising less than 0.2 mg/mL buffer, e.g., 0.08 mg/mL. In certain embodiments, the ready-to-infuse phenylephrine compositions of the disclosure, comprising less than 0.2 mg/mL buffer/buffering agent, upon storage for at least about 6 months under refrigerated storage conditions at about 2-8° C., exhibit an assay of phenylephrine or pharmaceutically acceptable salt thereof from about 90% to about 110% of the dose amount. In certain embodiments, such compositions, upon storage under refrigerated storage conditions comprise phenylephrine citrate product impurity of less than 0.2% of the dose amount and total impurity of less than 0.8% of the dose amount.

In certain embodiments, the disclosure provides stable ready-to-infuse phenylephrine compositions comprising less than 0.2 mg/mL buffer, e.g., 0.08 mg/mL. In certain embodiments, the ready-to-infuse phenylephrine compositions of the disclosure, comprising less than 0.2 mg/mL buffer/buffering agent, upon storage for at least about 6 months under freezing storage conditions at about −20° C.±5° C., exhibit an assay of phenylephrine or pharmaceutically acceptable salt thereof from about 90% to about 110% of the dose amount. In certain embodiments, such compositions, upon storage under freezing storage conditions comprise phenylephrine citrate product impurity of less than 0.2% of the dose amount and total impurity of less than 0.8% of the dose amount.

### Method of Manufacture

In certain embodiment, the present invention relates to a method for making a stable ready-to-infuse pharmaceutical composition of phenylephrine or a pharmaceutically acceptable salt thereof. The method comprises: 1) adding a tonicity agent to about 95% of the total volume of water for injection (WFI) and mixing to obtain a clear solution: 2) adding buffer to the solution from step #1 and mixing to obtain a clear solution: 3) adding phenylephrine hydrochloride to the solution from step #2 and mixing to obtain a clear solution: 4) adding a pH adjusting agent to the solution from step #3 to obtain a pH of about 4.5 and mixing to obtain a pH adjusted solution: 5) adding remaining amount of WFI to the pH adjusted solution from step #4 to make up the volume to 100% and mixing to obtain a bulk solution: 6) filtering the bulk solution from step #5 to obtain a filtered solution: 7) filing the filtered solution from step #6 into a bag and stoppered with a twist off port: 8) terminally sterilizing the bag from step #7 to obtain a terminally sterilized product: 9) placing an oxygen scavenger on the bag from step #8 and overwrapping with an aluminum over pouch.

### Method of Treatment

In certain embodiments, the disclosure provides methods of using ready-to-infuse phenylephrine or a pharmaceutically acceptable salt thereof compositions for increasing blood pressure in adults with clinically important hypotension resulting primarily from vasodilation in the settings of anaesthesia and septic shock. In certain embodiments, the ready-to-infuse phenylephrine compositions of the disclosure comprise single dose IV bags suitable for single use only. In certain embodiments, the ready-to-infuse phenylephrine compositions of the disclosure comprise 20 mg/250 mL (20 mg phenylephrine hydrochloride/250 mL of 0.9% sodium chloride) single-use IV bags for intravenous infusion. In certain embodiments, the ready-to-infuse phenylephrine compositions of the disclosure comprise 40 mg/250 mL (40 mg phenylephrine hydrochloride/250 mL of 0.9% sodium chloride) single-dose IV bags for intravenous infusion. In certain embodiments, the ready-to-infuse phenylephrine compositions of the disclosure comprise 25 mg/250 mL (25 mg phenylephrine hydrochloride/250 mL of 0.9% sodium chloride) single-dose IV bags for intravenous infusion. In certain embodiments, the ready-to-infuse phenylephrine compositions of the disclosure comprise 100 mg/250 ml (100 mg phenylephrine hydrochloride/250 mL of 0.9% sodium chloride) single-dose IV bags for intravenous infusion. The compositions of the disclosure packed in a single-use IV bag cannot be reused after opening or penetration of the bag.

### Method of Administration

In certain embodiments, the compositions of the disclosure comprise phenylephrine hydrochloride in 0.9% sodium chloride injection supplied in an intravenous bag wrapped in aluminum over pouch and an oxygen scavenger placed between the bag and the over pouch. The compositions are suitable for continuous infusion as intravenous injection.

The ready-to-infuse injection solution is visually inspected for particulate matter and discoloration prior to administration. Coloured or cloudy solutions or solutions containing particulate matter are discarded. During administration intravascular volume depletion and acidosis is corrected.

### Dosing for Anaesthesia in Perioperative Setting

In adult patients undergoing surgical procedures with either neuraxial anaesthesia or general anaesthesia, 0.5 mcg/kg/min to 1.4 mcg/kg/min of phenylephrine or a salt thereof is administered by intravenous continuous infusion, titrated to blood pressure goal.

### Dosing for Septic or Other Vasodilatory Shock

In adult patients with septic or other vasodilatory shock, 0.5 mcg/kg/min to 6 mcg/kg/min of phenylephrine or a salt thereof is administered by intravenous continuous infusion, titrated to blood pressure goal. Doses above 6 mcg/kg/min do not show significant incremental increase in blood pressure.

### EXAMPLES

The following examples illustrate the disclosure in a nonlimiting manner. Unless indicated to the contrary, the numerical parameters set forth herein can vary depending upon the desired properties sought to be obtained by the present disclosure.

### Example 1: Compositions of Phenylephrine Hydrochloride in 0.9% Sodium Chloride Injection

TABLE 1

| | Composition | | | |
|---|---|---|---|---|
| | 1 | 2 | 3 | 4 |
| Ingredients | mg/mL | mg/mL | mg/mL | mg/mL |
| Phenylephrine | 0.08 | 0.1 | 0.16 | 0.4 |
| Hydrochloride | | | | |
| Sodium chloride | 9.00 | 9.00 | 9.00 | 9.00 |
| Sodium citrate | 0.064 | 0.064 | 0.064 | 0.064 |
| dihydrate | | | | |
| Citric acid | 0.016 | 0.016 | 0.016 | 0.016 |
| monohydrate | | | | |
| Hydrochloric acid | Q.S. to pH | Q.S. to pH | Q.S. to pH | Q.S. to pH |
| Sodium Hydroxide | Q.S. to pH | Q.S. to pH | Q.S. to pH | Q.S. to pH |
| Water for injection | Q.S. to 1 | Q.S. to 1 | Q.S. to 1 | Q.S. to 1 |
| | mL | mL | mL | mL |

### Process for Preparation:

1. About 95% of the total volume of water for injection (WFI) was taken in a manufacturing vessel.
2. Weighed quantity of sodium chloride was added to the WFI from step #1 and mixed to obtain a clear solution.
3. Weighed quantity of sodium citrate dihydrate and citric acid were added in the solution from step #2 and mixed to obtain a clear solution.
4. Weighed quantity of phenylephrine hydrochloride was added in the solution from step #3 and mixed to obtain a clear solution.
5. The pH of solution from step #5 was adjusted to a desired pH of about 4.5 with hydrochloric acid and sodium hydroxide.
6. The remaining amount of water for injection was added to the pH adjusted solution from step #5 to make up the volume to 100% of the total batch size. The final volume of was mixed to obtain a clear solution.
7. The clear solution from step #6 was filtered using a filter to obtain a filtered bulk solution.
8. The filtered bulk solution from step #7 was filled in a bag stoppered with a twist off port.
9. The bag filled from step 8 was terminally sterilized; and placed in aluminum over pouch along with oxygen scavenger between the bag and the over pouch.
10. Steps 1-9 were carried under continuous nitrogen purging.

### Example 2: Compositions of Phenylephrine Hydrochloride in 5% Dextrose Injection

TABLE 2

| | Composition | | | |
|---|---|---|---|---|
| | 1 | 2 | 3 | 4 |
| Ingredients | mg/mL | mg/mL | mg/mL | mg/mL |
| Phenylephrine | 0.08 | 0.1 | 0.16 | 0.4 |
| Hydrochloride | | | | |
| Dextrose | 50.0 | 50.0 | 50.0 | 50.0 |
| Sodium citrate | 0.064 | 0.064 | 0.064 | 0.064 |
| dihydrate | | | | |
| Citric acid | 0.016 | 0.016 | 0.016 | 0.016 |
| monohydrate | | | | |
| Hydrochloric acid | Q.S. to pH | Q.S. to pH | Q.S. to pH | Q.S. to pH |
| Sodium Hydroxide | Q.S. to pH | Q.S. to pH | Q.S. to pH | Q.S. to pH |
| Water for injection | Q.S. to 1 | Q.S. to 1 | Q.S. to 1 | Q.S. to 1 |
| | mL | mL | mL | mL |

### Example 3: Accelerated Storage Stability of Phenylephrine Hydrochloride in 0.9% Sodium Chloride Injection Composition

Storage stability for 3 batches of phenylephrine hydrochloride Composition 1 was determined under accelerated storage conditions (40° C.±2° C./NMT 25% RH).

Table 3 provides 6-month storage stability of Batches 1-3 of terminally sterilized Composition 1 in a bag with aluminum over pouch and oxygen scavenger placed between the bag and the over pouch, under accelerated storage conditions.

TABLE 3
Phenylephrine Hydrochloride in 0.9% Sodium Chloride
Injection, 0.08 mg/mL (20 mg/250 mL) (Accelerated)

| Batch No. | | Batch 1 | | | Batch 2 | | | Batch 3 | | |
|---|---|---|---|---|---|---|---|---|---|---|
| Test | Specification Limit/Station | Initial | 3 M | 6 M | Initial | 3 M | 6 M | Initial | 3 M | 6 M |
| Description | Clear, Colorless solution | Complies | | | | | | | | |
| | filled in bags | | | | | | | | | |
| pH | Between 3.0 and 6.5 | 4.5 | 4.5 | 4.7 | 4.5 | 4.5 | 4.5 | 4.5 | 4.5 | 4.5 |
| Assay of | Between 90% and 110% | 100 | 101 | 101 | 100 | 101 | 101 | 100 | 100 | 100 |
| Phenylephrine | of dose amount | | | | | | | | | |
| Hydrochloride | | | | | | | | | | |
| Osmolality | Between 260 and 330 | 290 | 290 | 287 | 287 | 289 | 286 | 287 | 289 | 286 |
| | mOsmol/Kg | | | | | | | | | |
| Related | Phenylephrine Citrate | ND | ND | ND | ND | ND | ND | ND | ND | ND |
| Compound | Adduct^{a}: NMT 0.2% | | | | | | | | | |
| | Phenylephrine RC F^{b}: | BQL | BQL | BQL | BQL | BQL | BQL | BQL | BDL | BQL |
| | NMT 0.2% | | | | | | | | | |
| | Norphenylephrine^{c}: NMT | ND | ND | ND | ND | ND | ND | ND | ND | ND |
| | 0.2% | | | | | | | | | |
| | Phenylephrine RC C^{d}: | ND | ND | ND | ND | ND | ND | ND | ND | ND |
| | NMT 0.2% | | | | | | | | | |
| | Any Unspecified | 0.1 | 0.1 | 0.1 | 0.1 | 0.1 | 0.1 | 0.1 | 0.1 | 0.1 |
| | impurity: NMT 0.2% | | | | | | | | | |
| | Total impurities: NMT | 0.1 | 0.1 | 0.1 | 0.1 | 0.1 | 0.1 | 0.1 | 0.1 | 0.1 |
| | 0.8% | | | | | | | | | |

ND: Not detected; NMT: Not more than; BQL: Below quantification limit (<0.04% & >0.02%); BDL: Below Detection limit (<0.02%); RC: Related Compound.
^{a}2-Hydroxy-2-(2-{[(R)-2-hydroxy-2-(3-hydroxyphenyl)ethyl](methyl)amino}-2 oxoethyl)succinic acid.
^{b}2-Methyl-1,2,3,4-tetrahydroisoquinoline-4,8-diol.
^{c}3-(2-Amino-1-hydroxyethyl)phenol.
^{d}1-(3-Hydroxyphenyl)-2-(methylamino)ethan-1-one hydrochloride.

Table 4 provides 6-month storage stability of Batches 1-3 of terminally sterilized Composition 3 in a bag with aluminum over pouch and oxygen scavenger placed between the bag and the over pouch, under accelerated storage conditions (40° C.)±2° C./NMT 25% RH).

TABLE 4
Phenylephrine Hydrochloride in 0.9% Sodium Chloride
Injection, 0.16 mg/mL (40 mg/250 mL) (Accelerated)

| Batch No. | | | | | | | | | | |
|---|---|---|---|---|---|---|---|---|---|---|
| | Specification | Batch 1 | | | Batch 2 | | | Batch 3 | | |
| Test | Limit/Station | Initial | 3 M | 6 M | Initial | 3 M | 6 M | Initial | 3 M | 6 M |
| Description | Clear, Colorless | Complies | | | | | | | | |
| | solution filled | | | | | | | | | |
| | in bags | | | | | | | | | |
| pH | Between 3.0 and | 4.5 | 4.5 | 4.5 | 4.6 | 4.5 | 4.5 | 4.5 | 4.5 | 4.5 |
| | 6.5 | | | | | | | | | |
| Assay of | Between 90% and | 102 | 101 | 101 | 101 | 101 | 101 | 101 | 102 | 101 |
| Phenylephrine | 110% of dose | | | | | | | | | |
| Hydrochloride | amount | | | | | | | | | |
| Osmolality | Between 260 and | 290 | 292 | 288 | 287 | 290 | 287 | 290 | 308 | 287 |
| | 330 mOsmol/Kg | | | | | | | | | |
| Related | Phenylephrine | ND | ND | ND | ND | ND | ND | ND | ND | ND |
| Compound | Citrate Adduct: | | | | | | | | | |
| | NMT 0.2% | | | | | | | | | |
| | Phenylephrine RC | BDL | ND | ND | BDL | ND | BDL | BDL | ND | BDL |
| | F: NMT 0.2% | | | | | | | | | |
| | Norphenylephrine: | ND | ND | ND | ND | ND | ND | ND | ND | ND |
| | NMT 0.2% | | | | | | | | | |
| | Phenylephrine RC | ND | ND | ND | ND | ND | ND | ND | ND | ND |
| | C: NMT 0.2% | | | | | | | | | |
| | Any Unspecified | 0.1 | 0.1 | 0.1 | 0.1 | 0.1 | 0.1 | 0.1 | 0.1 | 0.1 |
| | impurity: NMT | | | | | | | | | |
| | 0.2% | | | | | | | | | |
| | Total impurities: | 0.1 | 0.1 | 0.1 | 0.1 | 0.1 | 0.1 | 0.1 | 0.1 | 0.1 |
| | NMT 0.8% | | | | | | | | | |

ND: Not detected; NMT: Not more than; BQL: Below quantification limit (<0.04% & >0.02%); BDL: Below Detection limit (<0.02%); RC: Related Compound.

Data from Tables 3 and 4 demonstrates excellent storage stability of the ready-to-infuse Composition 1 and 3 under accelerated storage conditions. The composition exhibits minimal batch-to-batch variability while maintaining the pH from 4.5-4.7; an assay of phenylephrine hydrochloride of from 100-102% of the dose amount; the phenylephrine citrate adduct remains not detectable (i.e., NMT 0.02%) and the total impurities remains to be 0.1% during the 6-month storage under accelerated storage conditions.

### Example 4: Long-Term Storage Stability of Phenylephrine Hydrochloride in 0.9% Sodium Chloride Injection Composition

Storage stability for 3 batches of phenylephrine hydrochloride Composition 1 was determined under long-term storage conditions (25° C.±2° C./40% RH±5% RH).

Table 5-7 provides 12-month storage stability of Batch 1-3 of terminally sterilized Composition 1 in a bag with aluminum over pouch and oxygen scavenger placed between the bag and the over pouch under long-term storage conditions.

TABLE 5
Phenylephrine Hydrochloride in 0.9% Sodium Chloride
Injection, 0.08 mg/mL (20 mg/250 mL) (Long-Term)

| Batch No. | | | | | | |
|---|---|---|---|---|---|---|
| | Specification Limit/ | Batch 1 | | | | |
| Test | Station | Initial | 3 M | 6 M | 9 M | 12 M |
| Description | Clear, Colorless solution | Complies | | | | |
| | filled in bags | | | | | |
| pH | Between 3.0 and 6.5 | 4.5 | 4.5 | 4.5 | 4.6 | 4.9 |
| Assay of | Between 90% and 110% | 100 | 101 | 100 | 101 | 101 |
| Phenylephrine | of dose amount | | | | | |
| Hydrochloride | | | | | | |
| Osmolality | Between 260 and 330 | 290 | 290 | 286 | 287 | 288 |
| | mOsmol/Kg | | | | | |
| Related | Phenylephrine Citrate | ND | ND | ND | ND | ND |
| Compound | Adduct: NMT 0.2% | | | | | |
| | Phenylephrine RC F: | BQL | BQL | BQL | BQL | ND |
| | NMT 0.2% | | | | | |
| | Norphenylephrine: NMT | ND | ND | ND | BDL | ND |
| | 0.2% | | | | | |
| | Phenylephrine RC C: | ND | ND | ND | ND | ND |
| | NMT 0.2% | | | | | |
| | Any Unspecified | 0.1 | 0.1 | 0.1 | 0.1 | 0.1 |
| | impurity: NMT 0.2% | | | | | |
| | Total impurities: NMT | 0.1 | 0.1 | 0.1 | 0.1 | 0.1 |
| | 0.8% | | | | | |

ND: Not detected; NMT: Not more than; BQL: Below quantification limit (<0.04% & >0.02%); BDL: Below Detection limit (<0.02%); RC: Related Compound.

TABLE 6
Phenylephrine Hydrochloride in 0.9% Sodium Chloride
Injection, 0.08 mg/mL (20 mg/250 mL) (Long-Term)

| Batch No. | | | | | | |
|---|---|---|---|---|---|---|
| | Specification Limit/ | Batch 2 | | | | |
| Test | Station | Initial | 3 M | 6 M | 9 M | 12 M |
| Description | Clear, Colorless solution | Complies | | | | |
| | filled in bags | | | | | |
| pH | Between 3.0 and 6.5 | 4.5 | 4.5 | 4.5 | 4.5 | 4.6 |
| Assay of | Between 90% and 110% | 100 | 101 | 101 | 101 | 101 |
| Phenylephrine | of dose amount | | | | | |
| Hydrochloride | | | | | | |
| Osmolality | Between 260 and 330 | 287 | 290 | 286 | 287 | 289 |
| | mOsmol/Kg | | | | | |
| Related | Phenylephrine Citrate | ND | ND | ND | ND | ND |
| Compound | Adduct: NMT 0.2% | | | | | |
| | Phenylephrine RC F: | BQL | ND | ND | BDL | ND |
| | NMT 0.2% | | | | | |
| | Norphenylephrine: NMT | ND | ND | ND | ND | ND |
| | 0.2% | | | | | |
| | Phenylephrine RC C: | ND | ND | ND | ND | ND |
| | NMT 0.2% | | | | | |
| | Any Unspecified | 0.1 | 0.1 | 0.1 | 0.1 | 0.1 |
| | impurity: NMT 0.2% | | | | | |
| | Total impurities: NMT | 0.1 | 0.1 | 0.1 | 0.1 | 0.1 |
| | 0.8% | | | | | |

ND: Not detected; NMT: Not more than; BQL: Below quantification limit (<0.04% & >0.02%); BDL: Below Detection limit (<0.02%); RC: Related Compound.

TABLE 7
Phenylephrine Hydrochloride in 0.9% Sodium Chloride
Injection, 0.08 mg/mL (20 mg/250 mL) (Long-Term)

| Batch No. | | | | | | |
|---|---|---|---|---|---|---|
| | Specification Limit/ | Batch 3 | | | | |
| Test | Station | Initial | 3 M | 6 M | 9 M | 12 M |
| Description | Clear, Colorless solution | Complies | | | | |
| | filled in bags | | | | | |
| pH | Between 3.0 and 6.5 | 4.5 | 4.5 | 4.5 | 4.5 | 4.7 |
| Assay of | Between 90% and 110% | 100 | 100 | 100 | 101 | 100 |
| Phenylephrine | of dose amount | | | | | |
| Hydrochloride | | | | | | |
| Osmolality | Between 260 and 330 | 287 | 288 | 286 | 287 | 288 |
| | mOsmol/Kg | | | | | |
| Related | Phenylephrine Citrate | ND | ND | ND | ND | ND |
| Compound | Adduct: NMT 0.2% | | | | | |
| | Phenylephrine RC F: NMT | BQL | ND | BDL | BDL | ND |
| | 0.2% | | | | | |
| | Norphenylephrine: NMT | ND | ND | ND | ND | ND |
| | 0.2% | | | | | |
| | Phenylephrine RC C: NMT | ND | ND | ND | ND | ND |
| | 0.2% | | | | | |
| | Any Unspecified impurity: | 0.1 | 0.1 | 0.1 | 0.1 | 0.1 |
| | NMT 0.2% | | | | | |
| | Total impurities: NMT | 0.1 | 0.1 | 0.1 | 0.1 | 0.1 |
| | 0.8% | | | | | |

ND: Not detected; NMT: Not more than; BQL: Below quantification limit (<0.04% & >0.02%); BDL: Below Detection limit (<0.02%); RC: Related Compound.

Data from Table 5-7 demonstrates excellent storage stability, with minimal batch-to-batch variability of the ready-to-use Composition 1 under long-term storage conditions. The compositions maintain the pH from 4.5-4.9 during the 12-month storage under long-term storage conditions; maintain an assay of phenylephrine hydrochloride from 100-101% of the dose amount; the phenylephrine citrate adduct remains not detectable (i.e., NMT 0.02%) and the total impurities remains to be 0.1% during the 12-month storage under long-term storage conditions.

Table 8-10 provides 12-month storage stability of Batch 1-3 of terminally sterilized Composition 3 in a bag with aluminum over pouch and oxygen scavenger placed between the bag and the over pouch, under long-term storage conditions (25° C.±2° C./40% RH±5% RH).

TABLE 8
Phenylephrine Hydrochloride in 0.9% Sodium Chloride
Injection, 0.16 mg/mL (40 mg/250 mL) (Long-Term)

| Batch No. | | | | | | |
|---|---|---|---|---|---|---|
| | Specification Limit/ | Batch 1 | | | | |
| Test | Station | Initial | 3 M | 6 M | 9 M | 12 M |
| Description | Clear, Colorless | Complies | | | | |
| | solution filled in bags | | | | | |
| pH | Between 3.0 and 6.5 | 4.5 | 4.5 | 4.5 | 4.5 | 4.5 |
| Assay of | Between 90% and 110% | 102 | 101 | 101 | 101 | 101 |
| Phenylephrine | of dose amount | | | | | |
| Hydrochloride | | | | | | |
| Osmolality | Between 260 and 330 | 290 | 290 | 287 | 290 | 291 |
| | mOsmol/Kg | | | | | |
| Related | Phenylephrine Citrate | ND | ND | ND | ND | ND |
| Compound | Adduct: NMT 0.2% | | | | | |
| | Phenylephrine RC F: | BDL | ND | ND | ND | ND |
| | NMT 0.2% | | | | | |
| | Norphenylephrine: NMT | ND | ND | ND | ND | ND |
| | 0.2% | | | | | |
| | Phenylephrine RC C: | ND | ND | ND | ND | ND |
| | NMT 0.2% | | | | | |
| | Any Unspecified | 0.1 | 0.1 | 0.1 | 0.1 | 0.1 |
| | impurity: NMT 0.2% | | | | | |
| | Total impurities: NMT | 0.1 | 0.1 | 0.1 | 0.1 | 0.1 |
| | 0.8% | | | | | |

ND: Not detected; NMT: Not more than; BQL: Below quantification limit (<0.04% & >0.02%); BDL: Below Detection limit (<0.02%); RC: Related Compound.

TABLE 9
Phenylephrine Hydrochloride in 0.9% Sodium Chloride
Injection, 0.16 mg/mL (40 mg/250 mL) (Long-Term)

| Batch No. | | | | | | |
|---|---|---|---|---|---|---|
| | Specification Limit/ | Batch 2 | | | | |
| Test | Station | Initial | 3 M | 6 M | 9 M | 12 M |
| Description | Clear, Colorless | Complies | | | | |
| | solution filled in bags | | | | | |
| pH | Between 3.0 and 6.5 | 4.6 | 4.5 | 4.5 | 4.5 | 4.6 |
| Assay of | Between 90% and | 101 | 101 | 101 | 101 | 101 |
| Phenylephrine | 110% of dose | | | | | |
| Hydrochloride | amount | | | | | |
| Osmolality | Between 260 and 330 | 287 | 289 | 288 | 287 | 289 |
| | mOsmol/Kg | | | | | |
| Related | Phenylephrine | ND | ND | ND | ND | ND |
| Compound | Citrate Adduct: NMT | | | | | |
| | 0.2% | | | | | |
| | Phenylephrine RC F: | BDL | ND | BDL | BDL | ND |
| | NMT 0.2% | | | | | |
| | Norphenylephrine: | ND | ND | ND | ND | ND |
| | NMT 0.2% | | | | | |
| | Phenylephrine RC C: | ND | ND | ND | ND | ND |
| | NMT 0.2% | | | | | |
| | Any Unspecified | 0.1 | 0.1 | 0.1 | 0.1 | 0.1 |
| | impurity: NMT 0.2% | | | | | |
| | Total impurities: | 0.1 | 0.1 | 0.1 | 0.1 | 0.1 |
| | NMT 0.8% | | | | | |

ND: Not detected; NMT: Not more than; BQL: Below quantification limit (<0.04% & >0.02%); BDL: Below Detection limit (<0.02%); RC: Related Compound.

TABLE 10
Phenylephrine Hydrochloride in 0.9% Sodium Chloride
Injection, 0.16 mg/mL (40 mg/250 mL) (Long-Term)

| Batch No. | | | | | | |
|---|---|---|---|---|---|---|
| | Specification Limit/ | Batch 3 | | | | |
| Test | Station | Initial | 3 M | 6 M | 9 M | 12 M |
| Description | Clear, Colorless | Complies | | | | |
| | solution filled in bags | | | | | |
| pH | Between 3.0 and 6.5 | 4.5 | 4.5 | 4.5 | 4.5 | 4.5 |
| Assay of | Between 90% and | 101 | 102 | 101 | 101 | 101 |
| Phenylephrine | 110% of dose | | | | | |
| Hydrochloride | amount | | | | | |
| Osmolality | Between 260 and 330 | 290 | 294 | 287 | 290 | 289 |
| | mOsmol/Kg | | | | | |
| Related | Phenylephrine | ND | ND | ND | ND | ND |
| Compound | Citrate Adduct: NMT | | | | | |
| | 0.2% | | | | | |
| | Phenylephrine RC F: | BDL | ND | BDL | BDL | ND |
| | NMT 0.2% | | | | | |
| | Norphenylephrine: | ND | ND | ND | ND | ND |
| | NMT 0.2% | | | | | |
| | Phenylephrine RC C: | ND | ND | ND | ND | ND |
| | NMT 0.2% | | | | | |
| | Any Unspecified | 0.1 | 0.1 | 0.1 | 0.1 | 0.1 |
| | impurity: NMT 0.2% | | | | | |
| | Total impurities: | 0.1 | 0.1 | 0.1 | 0.1 | 0.1 |
| | NMT 0.8% | | | | | |

ND: Not detected; NMT: Not more than; BQL: Below quantification limit (<0.04% & >0.02%); BDL: Below Detection limit (<0.02%); RC: Related Compound.

Data from Table 8-10 demonstrates excellent storage stability, with minimal batch-to-batch variability of the ready-to-use Composition 3 under long-term storage conditions. The composition maintains the pH from 4.5-4.6 during the 12-month storage under long-term storage conditions; maintain an assay of phenylephrine hydrochloride from 101-102% of the dose amount; the phenylephrine citrate adduct remains not detectable (i.e., NMT 0.02%) and the total impurities remains to be 0.1% during the 12-month storage under long-term storage conditions.

### Example 5: Effect of the Presence of Oxygen Scavenger on Accelerated Storage Stability of Terminally Sterilized Phenylephrine Hydrochloride in 0.9% Sodium Chloride Injection Composition

To study the impact of oxygen scavenger, stability of Composition 1 was evaluated under accelerated storage conditions for 1 month. Table 11 provides stability data of Composition 1 with and without oxygen scavenger.

TABLE 11

| | Specification | With Oxygen scavenger | | Without Oxygen scavenger | |
|---|---|---|---|---|---|
| Tests | Limit/Station | Initial | 1 M 40° C./25% RH | Initial | 1 M 40° C./25% RH |
| Description | Clear, Colorless | Complies | | | |
| | solution filled | | | | |
| | in bags | | | | |
| pH | Between 3.0 | 4.5 | 4.6 | 4.5 | 4.5 |
| | and 6.5 | | | | |
| Assay of | Between 90% | 99.1 | 102 | 101 | 102 |
| Phenylephrine | and 110% of | | | | |
| Hydrochloride | dose amount | | | | |
| Total | Total | 0.34% | 0.32% | 0.25% | 0.62% |
| Impurities | impurities: | | | | |
| | NMT 0.8% | | | | |

Data from Table 11 demonstrates that oxygen scavenger has a significant impact on total impurities. Composition 1 without oxygen scavenger resulted in total impurities of 0.62% as compared to with oxygen scavenger having total impurities of 0.32%.

### Example 6: Storage Stability of Phenylephrine Hydrochloride in 0.9% Sodium Chloride Injection Composition Under Refrigerated Storage Conditions

Table 12 provide 6-month storage stability under refrigerated storage conditions (5° C.±3° C.) for phenylephrine hydrochloride Compositions 1 and 3.

TABLE 12

| Tests | Composition 1 | Composition 3 |
|---|---|---|
| pH | 4.6 | 4.6 |
| Osmolality (mOsmol/Kg) | 289 | 289 |
| Assay of Phenylephrine Hydrochloride | 99.6% | 100% |
| Phenylephrine Citrate adduct | ND | ND |
| Phenylephrine RC F | 0.08% | 0.12% |
| Norphenylephrine | ND | ND |
| Phenylephrine RC C | ND | ND |
| Total Impurities | NMT 0.24% | NMT 0.33% |

ND: Not detected; NMT: Not more than; RC: Related Compound.

Data from Table 12 demonstrates excellent storage stability of the ready-to-use Compositions 1 and 3 under refrigerated storage conditions. The compositions exhibit a pH of about 4.6; maintain an assay of phenylephrine hydrochloride from about 100% of the dose amount: phenylephrine citrate adduct remains not detectable (i.e., NMT 0.02%) and total impurities of less than 0.4%, on 6-month-month storage under refrigerated storage conditions.

### Example 7: Storage Stability of Phenylephrine Hydrochloride in 0.9% Sodium Chloride Injection Composition Under Freezing Storage Conditions

Table 13 provide 6-month storage stability under freezing storage conditions (−20° C.±5° C.) for phenylephrine hydrochloride Compositions 1 and 3.

TABLE 13

| Tests | Composition 1 | Composition 3 |
|---|---|---|
| pH | 4.6 | 4.6 |
| Osmolality (mOsmol/Kg) | 289 | 290 |
| Assay of Phenylephrine Hydrochloride | 99.4% | 99.8% |
| Phenylephrine Citrate adduct | ND | ND |
| Pheny lephrine RC F | 0.08% | 0.05% |
| Norphenylephrine | ND | ND |
| Pheny lephrine RC C | ND | ND |
| Total Impurities | NMT 0.32% | NMT 0.26% |

ND: Not detected; NMT: Not more than; RC: Related Compound.

Data from Table 13 demonstrates excellent storage stability of the ready-to-use Compositions 1 and 3 under freezing storage conditions. The compositions exhibit a pH of about 4.6; maintain an assay of phenylephrine hydrochloride from about 100% of the dose amount; phenylephrine citrate adduct remains not detectable (i.e., NMT 0.02%) and total impurities of less than 0.3%, on 6-month-month storage under freezing storage conditions.

1. A ready-to-infuse, inert gas purged phenylephrine injection solution comprising a dose amount of phenylephrine or a pharmaceutically acceptable salt thereof, a pH adjusting agent, and a citrate buffer in an amount of from about 0.0001 mg/mL to about 0.15 mg/mL; wherein the solution is manufactured and packaged under inert gas purging and exhibits a pH of from 4.0-4.6; and wherein the solution upon storage for at least about 6 months at 25° C.±2° C./40%±5% relative humidity does not contain more than 0.2% wt/vol of phenylephrine citrate adduct impurity, based on the dose amount of the phenylephrine or-a pharmaceutically acceptable salt thereof.

2. The injection solution of claim 1 comprising from about 0.01 mg/mL to about 1 mg/mL of phenylephrine hydrochloride.

3. The injection solution of claim 1, wherein the citrate buffer comprises 0.064 mg/mL sodium citrate and 0.016 mg/mL citric acid.

4. The injection solution of claim 1, wherein the pH adjusting agent is an acidic pH adjusting agent and a basic pH adjusting agent.

5. The injection solution of claim 1, wherein the solution upon storage for at least about 6 months at 25° C.±2° C./40%±5% relative humidity maintains the pH of from about 4.0-4.6.

6. (canceled)

7. The injection solution of claim 1, wherein the solution is free of disodium edetate.

8. The injection solution of claim 1, wherein the solution is supplied in a flexible plastic container packed in aluminium over pouch, and an oxygen scavenger between the bag and the aluminum over pouch.

9. A ready-to-infuse, inert gas purged phenylephrine injection solution comprising a dose amount of phenylephrine or a pharmaceutically acceptable salt thereof, a pH adjusting agent, and a citrate buffer in an amount of from about 0.0001 mg/mL to about 0.15 mg/mL; wherein the solution is manufactured and packaged under inert gas purging and exhibits a pH of from 4.0-4.6; and wherein the solution upon storage for about 6 months at 40° C.±2° C./not more than 25% relative humidity does not contain more than 0.2% wt/vol of phenylephrine citrate adduct impurity, based on the dose amount of the phenylephrine or a pharmaceutically acceptable salt thereof.

10. The injection solution of claim 9 comprising from about 0.01 mg/mL to about 1 mg/mL of phenylephrine hydrochloride.

11. The injection solution of claim 9, wherein the citrate buffer comprises 0.064 mg/mL sodium citrate and 0.016 mg/mL citric acid.

12. The injection solution of claim 9, wherein the pH adjusting agent is an acidic pH adjusting agent and a basic pH adjusting agent.

13. The injection solution of claim 9, wherein the solution, upon storage for at least about 6 months at 40° C.±2° C./not more than 25% relative humidity, maintains the pH of from about 4.0-4.6.

14. (canceled)

15. The injection solution of claim 9, wherein the solution is free of disodium edetate.

16. The injection solution of claim 9, wherein the solution is supplied in a flexible plastic container packed in aluminium over pouch, and an oxygen scavenger between the bag and the aluminum over pouch.

17. A ready-to-infuse, inert gas purged phenylephrine injection solution in a flexible plastic container packed in aluminium over pouch, and an oxygen scavenger between the bag and the aluminum over pouch, wherein the solution comprises 0.08 mg/mL or 0.16 mg/mL of phenylephrine hydrochloride, a pH adjusting agent, and a citrate buffer in an amount of from about 0.0001 mg/mL to about 0.15 mg/mL; wherein the solution is manufactured and packaged under inert gas purging and exhibits a pH of from 4.0-4.6; and wherein the solution upon storage for at least about 6 months under freezing storage condition does not contain more than 0.2% wt/vol of phenylephrine citrate adduct impurity, based on the dose amount of the phenylephrine hydrochloride; wherein the freezing storage condition comprises a temperature of about −20° C.±5° C.

18. The injection solution of claim 17 comprising 0.016 mg/mL of citric acid, and 0.064 mg/mL of sodium citrate.

19. The injection solution of claim 17, wherein the solution is free of disodium edetate.

20. The injection solution of claim 17, wherein the pH adjusting agent is an acidic pH adjusting agent and a basic pH adjusting agent.

21. The injection solution of claim 17, wherein the inert gas is nitrogen.
